(19) United States
(12) Patent Application Publication
(10) Pub. No.: US 2022/0105278 A1
(43) Pub. Date: Apr. 7, 2022

(54) RIGID NEEDLE SHIELD REMOVER FOR AUTOINJECTOR

(71) Applicant: Becton Dickinson France, Le Pont-De-Claix (FR)

(72) Inventor: Louis Petit, Venon (FR)

(21) Appl. No.: 17/425,958

(22) PCT Filed: Feb. 26, 2020
(86) PCT No.: PCT/EP20/55012

§ 371 Date: Jul. 27, 2021

(30) Foreign Application Priority Data
Feb. 26, 2019 (EP) 19305228.9

(51) Int. Cl. A61M 5/32; A61M 5/20

(52) U.S. Cl. CPC A61M 5/3204; A61M 5/20; A61M 2207/00; A61M 2005/2006; A61M 2005/3254

(57) ABSTRACT

Provided herein is a shield removal assembly for an auto injector including a cap remover configured to be grasped by a user, a retainer received within the cap remover and having at least one grasping feature configured to engage a proximal edge of an auto injector needle shield. The shield removal assembly is configured such that the needle shield of the auto injector can be advanced through the shield removal assembly from a first position in which the at least one grasping feature is located proximally of the proximal edge of the needle shield to a second position in which the at least one grasping feature abuts the proximal edge of the needle shield.

### CROSS-REFERENCE TO RELATED APPLICATIONS

This application is the United States national phase of International Application No. PCT/EP2020/055012 filed Feb. 26, 2020, and claims priority to Europe patent Application No. 19305228.9 filed Feb. 26, 2019, the entire disclosures of which are hereby incorporated by reference in their entirety.

### BACKGROUND OF THE INVENTION

### Field of the Invention

The present disclosure relates generally to autoinjectors and methods of assembling the same and, more specifically, to devices for removing needle shields from autoinjectors and methods for assembling autoinjectors including such devices.

### Description of Related Art

Automatic drug delivery devices, such as autoinjectors, are designed with removable shields to maintain sterility of the needle and to prevent inadvertent contact between the needle and a user. Many of these needle shields are elastomeric, and ensuring accurate placement and a firm grip between the needle shield and caps or other removal devices can be difficult, resulting in a need for excess force to remove the shield or slippage and an inability to remove the shield at all. Accordingly, a need exists in the art for a needle shield remover that can reliably be positioned for quick and easy removal of the needle shield when the autoinjector is to be used.

### SUMMARY OF THE INVENTION

Provided herein is a shield removal assembly for an autoinjector including a syringe barrel, a needle, and a needle shield at least partially surrounding the needle. The shield removal assembly includes a cap remover configured to be grasped by a user and having a proximal end and a distal end, the distal end of the cap remover having at least one opening therethrough, and a retainer received within the cap remover and having a proximal end and a distal end, the retainer having a at least one grasping feature configured to engage at least one complementary grasping feature of a needle shield, and the distal end having an opening therethrough and configured to abut the distal end of the cap remover. The shield removal assembly is configured such that the needle shield of the autoinjector can be advanced through the shield removal assembly from a first position, in which the grasping feature is located proximally of the complementary grasping feature of the needle shield, to a second position, in which the grasping feature abuts the complementary grasping feature of the needle shield.

Also provided herein is an autoinjector including a housing having a proximal end, an open distal end, and defining a longitudinal axis, a syringe held within the housing and having a barrel, a needle attached to a distal end of the syringe barrel, a removable needle shield having a proximal end and a distal end, the needle shield configured to at least partially surround the needle, and a shield removal assembly.

Also provided herein is a method of assembling an autoinjector including the steps of providing a lower assembly including a lower housing having a proximal end and a distal end, and defining a longitudinal axis and a shield removal assembly arranged at the distal end of the lower housing and including a cap remover configured to be grasped by a user and having a proximal end, a distal end, the distal end of the cap remover having at least one opening therethrough, and a retainer received within the cap remover and having a proximal end having at least one grasping feature and a distal end, the distal end of the retainer comprising at least one opening therethrough and spaced from the distal end of the cap remover in an initial configuration. The method further includes inserting a syringe into the lower assembly, the syringe including a barrel having a proximal end and a distal end, a needle arranged at the distal end of the barrel, and a removable needle shield having a proximal end having at least one complementary grasping feature and a distal end, the needle shield configured to at least partially surround the needle. The step of inserting a syringe comprises moving the syringe towards the distal end of the lower housing along the longitudinal axis such that the grasping feature of the retainer contacts the syringe barrel proximal to the distal end of the syringe barrel and the distal end of the needle shield abuts the distal end of the retainer. The method further includes the step of placing an upper assembly comprising an upper housing on the lower assembly.

Further embodiments and aspects are set forth below:

A shield removal assembly for an autoinjector comprising a syringe barrel, a needle, and a needle shield at least partially surrounding the needle, comprising: a cap remover configured to be grasped by a user and having a proximal end and a distal end, the distal end of the cap remover comprising at least one opening therethrough; and a retainer received within the cap remover and having a proximal end and a distal end, the retainer comprising at least one grasping feature configured to engage at least one complementary grasping feature of a needle shield, and the distal end comprising an opening therethrough and configured to abut the distal end of the cap remover, wherein the shield removal assembly is configured such that the needle shield of the autoinjector can be advanced through the shield removal assembly from a first position in which the grasping feature is located proximally of the complementary grasping feature of the needle shield to a second position in which the grasping feature abuts the complementary grasping feature of the needle shield.

The shield removal assembly described above, wherein the grasping feature is a projection located at the proximal end of the retainer, the shield removal assembly configured such that the needle shield of the autoinjector can be advanced through the shield removal assembly from a first position in which the proximal end of the needle shield is longitudinally spaced from the proximal end) of the retainer to a second position in which the proximal end of the needle shield is longitudinally spaced from the proximal end of the retainer to a second position in which the proximal end of the needle shield abuts the proximal end of the retainer.

The shield removal assembly described above, wherein the retainer comprises at least one radially-extending protrusion, and the cap remover comprises at least one shoulder configured to engage the radially-extending protrusion.

The shield removal assembly described above, wherein the at least one radially-extending protrusion is longitudinally spaced from the shoulder, such that the cap remover is moved longitudinally away from the syringe a set distance before the radially extending protrusion of the retainer engages the shoulder in the cap remover.

The shield removal assembly described above, wherein the opening in the distal end of the cap remover has a greater diameter than the opening in the distal end of the retainer.

An autoinjector comprising: a housing having a proximal end, an open distal end, and defining a longitudinal axis; a syringe held within the housing and having a barrel; a needle attached to a distal end of the syringe barrel; a removable needle shield having a proximal end and a distal end, the needle shield configured to at least partially surround the needle; and a shield removal assembly as described above.

The autoinjector described above, wherein the cap remover at least partially surrounds the open distal end of the housing.

The autoinjector described above, wherein the cap remover is not rotatable relative to the housing.

A method of assembling an autoinjector comprising: providing a lower assembly, the lower assembly comprising: a lower housing having a proximal end and a distal end, and defining a longitudinal axis; and a shield removal assembly arranged at the distal end of the lower housing, comprising: a cap remover configured to be grasped by a user and having a proximal end, a distal end, the distal end of the cap remover comprising at least one opening therethrough; and a retainer received within the cap remover and having a proximal end having at least one grasping feature and a distal end, the distal end of the retainer comprising at least one opening therethrough and spaced from the distal end of the cap remover in an initial configuration; inserting a syringe into the lower assembly, the syringe comprising: a barrel having a proximal end and a distal end; a needle arranged at the distal end of the barrel; and a removable needle shield having a proximal end having at least one complementary grasping feature and a distal end, the needle shield configured to at least partially surround the needle, wherein the step of inserting comprises moving the syringe towards the distal end of the lower housing along the longitudinal axis such that: the grasping feature of the retainer contacts the syringe barrel proximal to the distal end of the syringe barrel; and the distal end of the needle shield abuts the distal end of the retainer; and placing an upper assembly comprising an upper housing on the lower assembly.

The method described above, wherein the grasping feature of the retainer comprises at least one projection configured to engage a proximal edge of the needle shield, and wherein the step of inserting the syringe into the lower assembly comprises urging the syringe distally such that the at least one projection is positioned proximally of the proximal edge of the needle shield.

The method described above, further comprising a step of, after inserting the syringe into the lower assembly, urging the syringe proximally such that the at least one grasping feature engages the proximal edge of the needle shield.

The method described above, wherein the step of urging the syringe proximally comprises inserting a device through the at least one opening in the distal end of the cap remover and the distal end of the retainer to urge the syringe proximally.

The method described above, wherein after urging the syringe proximally, the at least one projection engages the proximal edge of the needle shield.

The method described above, wherein the proximal end of the syringe barrel comprises a flange and the proximal end of the lower housing comprises a ring.

The method described above, wherein, after insertion of the syringe, the syringe flange abuts the ring.

The method described above, further comprising, after inserting the syringe into the lower assembly, a step of urging the syringe proximally, and wherein after the step of urging the syringe proximally, the syringe flange is longitudinally spaced from the ring.

The method described above, wherein the retainer comprises at least one radially-extending protrusion.

The method described above, wherein the cap remover comprises a shoulder in a sidewall thereof between the proximal end and the distal end of the cap remover.

An autoinjector comprising: a housing having a proximal end, an open distal end, and defining a longitudinal axis; a syringe held within the housing and having a barrel; a needle attached to a distal end of the syringe barrel; a removable needle shield having a proximal end and a distal end, the needle shield configured to at least partially surround the needle; and a shield removal assembly comprising: a cap remover configured to be grasped by a user and having a proximal end and a distal end, the distal end of the cap remover comprising at least one opening therethrough; and a retainer received within the cap remover and having a proximal end and a distal end, the proximal end configured to engage the needle shield between the proximal end of the needle shield and the distal end of the syringe barrel, and the distal end comprising at least one opening therethrough and configured to abut the distal end of the cap remover, wherein the needle shield is adapted to be advanced through the shield removal assembly from a first position in which the proximal end of the needle shield is longitudinally spaced from the proximal end of the retainer to a second position in which the proximal end of the needle shield abuts the proximal end of the retainer.

The autoinjector described above, wherein the retainer and the cap remover are arranged to provide lag between initiation of longitudinal movement of the cap remover and initiation of longitudinal movement of the retainer.

The autoinjector described above, wherein the proximal end of the retainer comprises at least one grasping feature.

The autoinjector described above, wherein, in the first position, the proximal end of the needle shield is longitudinally spaced from the at least one grasping feature and in the second position the proximal end of the needle shield abuts the at least one grasping feature.

The autoinjector described above, wherein the retainer comprises at least one member extending proximally from the distal end of the retainer, the at least one member comprising a radially-extending protrusion.

The autoinjector described above, wherein the cap remover at least partially surrounds the open distal end of the housing.

The autoinjector described above, wherein the cap remover is rotatably fixed relative to the housing.

A method of assembling an autoinjector comprising: providing a lower assembly, the lower assembly comprising: a lower housing having a proximal end and a distal end, and defining a longitudinal axis; and a shield removal assembly arranged at the distal end of the lower housing, comprising: a cap remover configured to be grasped by a user and having a proximal end and a distal end, the distal end of the cap remover comprising at least one opening therethrough; and a retainer received within the cap remover and having a proximal end and a distal end, the distal end of the retainer comprising at least one opening therethrough and spaced from the distal end of the cap remover in an initial configuration; inserting a syringe into the lower assembly, the syringe comprising: a barrel having a proximal end and a distal end; a needle arranged at the distal end of the barrel; and a removable needle shield having a proximal end and a distal end, the needle shield configured to at least partially surround the needle, wherein the step of inserting a syringe comprises moving the syringe towards the distal end of the lower housing along the longitudinal axis such that: the proximal end of the retainer contacts the syringe barrel proximal to the distal end of the syringe barrel; and the distal end of the needle shield abuts the distal end of the retainer; and placing an upper assembly comprising an upper housing on the lower assembly.

The method described above, wherein the proximal end of the retainer comprises at least one grasping feature, and wherein the step of inserting the syringe into the lower assembly comprises urging the syringe distally such that the at least one grasping feature contacts the syringe barrel proximally of the needle shield.

The method described above, further comprising a step of, after inserting the syringe into the lower assembly, urging the syringe proximally such that the at least one grasping feature is positioned between the distal end of the syringe barrel and the proximal end of the needle shield.

The method described above, wherein the step of urging the syringe proximally comprises inserting a device through the at least one opening in the distal end of the cap remover and the distal end of the retainer to urge the syringe proximally.

The method described above, wherein after urging the syringe proximally, the at least one grasping feature engages the proximal end of the needle shield.

The method described above, wherein the proximal end of the syringe barrel comprises a flange and the proximal end of the lower housing comprises a ring.

The method described above, wherein, after insertion of the syringe, the syringe flange abuts the ring.

The method described above, further comprising, after inserting the syringe into the lower assembly, a step of urging the syringe proximally, and wherein after the step of urging the syringe proximally, the flange is longitudinally spaced from the ring.

The method described above, wherein after the upper assembly is placed on the lower assembly, the flange abuts the ring.

The method described above, wherein the retainer comprises at least one member extending proximally from the distal end of the retainer, the at least one member comprising a radially-extending protrusion.

The method described above, wherein the cap remover comprises an opening in a sidewall thereof between the proximal end and the distal end of the cap remover.

A shield removal assembly for an autoinjector comprising a syringe barrel, a needle, and a needle shield at least partially surrounding the needle, comprising: a cap remover configured to be grasped by a user and having a proximal end and a distal end, the distal end of the cap remover comprising at least one opening therethrough; and a retainer received within the cap remover and having a proximal end and a distal end, the proximal end configured to engage the needle shield between the proximal end of the needle shield and the distal end of the syringe barrel, and the distal end comprising an opening therethrough and configured to abut the distal end of the cap remover, wherein the shield removal assembly is configured such that the needle shield of the autoinjector can be advanced through the shield removal assembly from a first position, in which the proximal end of the needle shield is longitudinally spaced from the proximal end of the retainer, to a second position, in which the proximal end of the needle shield abuts the proximal end of the retainer.

The shield removal assembly described above, wherein the retainer and the cap remover are arranged to provide lag between initiation of longitudinal movement of the cap remover and initiation of longitudinal movement of the retainer.

The shield removal assembly described above, wherein the retainer comprises at least one member extending proximally from the distal end of the retainer, the at least one member comprising a radially-extending protrusion.

The shield removal assembly described above, wherein the lag-providing arrangement defines a longitudinal distance between the radially-extending protrusion of the retainer and a shoulder of the cap remover, such that the cap remover is moved longitudinally away from the syringe a set distance before the radially extending protrusion of the retainer engages the shoulder of the cap remover.

The shield removal assembly described above, wherein the proximal end of the retainer comprises at least one grasping feature.

The shield removal assembly described above, wherein, in the first position, the proximal end of the needle shield is longitudinally spaced from the at least one grasping feature and, in the second position, the proximal end of the needle shield abuts the at least one grasping feature.

### BRIEF DESCRIPTION OF THE DRAWINGS

FIG. 1A is a side view of a shield removal assembly according to a non-limiting embodiment or aspect described herein;

FIG. 1B is a bottom view of a shield removal assembly according to a non-limiting embodiment or aspect described herein;

FIG. 2A is an exploded view of a shield removal assembly according to a non-limiting embodiment or aspect described herein;

FIG. 2B is a perspective view of the shield removal assembly as shown in FIG. 2A;

FIG. 3 is a cross-sectional view of a shield removal assembly according to a non-limiting embodiment or aspect described herein;

FIG. 4A is a perspective view of a shield removal assembly according to a non-limiting embodiment or aspect described herein;

FIG. 4B is a perspective view of a shield removal assembly according to a non-limiting embodiment or aspect described herein;

FIG. 5A is an exploded perspective view of a shield removal assembly according to a non-limiting embodiment or aspect described herein;

FIG. 5B is a perspective view of a cap remover of a shield removal assembly according to a non-limiting embodiment or aspect described herein;

FIG. 5C is a perspective view of a retainer of a shield removal assembly according to a non-limiting embodiment or aspect described herein;

FIG. 6 is a cross-sectional perspective view of a shield removal assembly according to a non-limiting embodiment or aspect described herein;

FIG. 7 is a cross-sectional view of an autoinjector including a shield removal assembly according to a non-limiting embodiment or aspect described herein;

FIG. 8 is a cross-sectional view of an autoinjector including a shield removal assembly according to a non-limiting embodiment or aspect described herein; and

FIG. 9 is a cross-sectional view of an autoinjector including a shield removal assembly according to a non-limiting embodiment or aspect described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

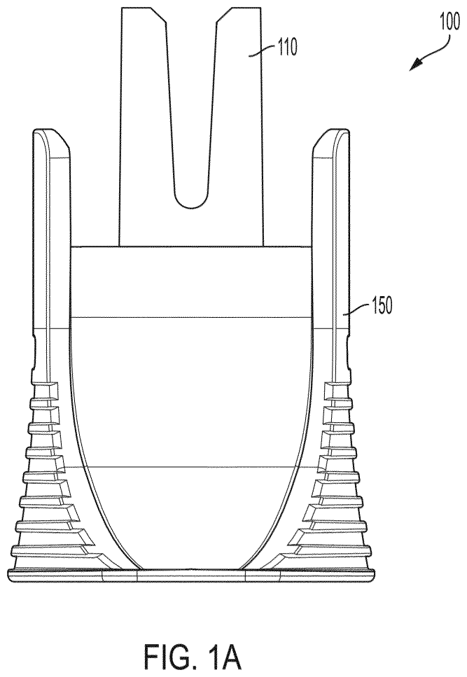

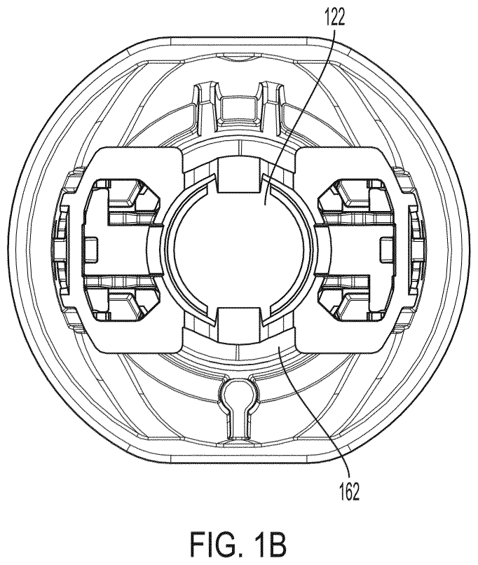

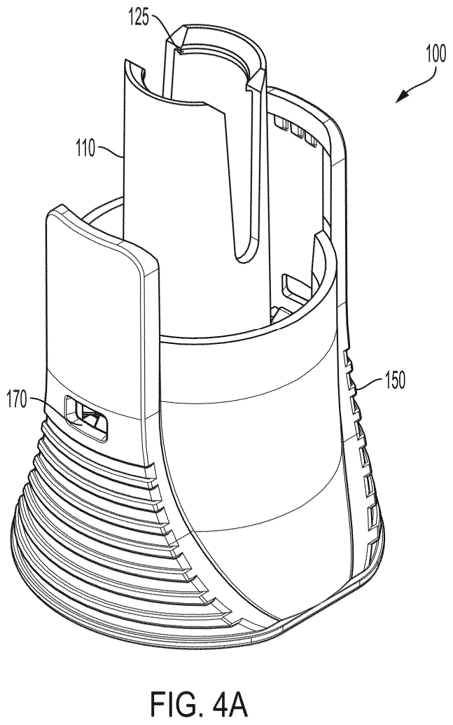

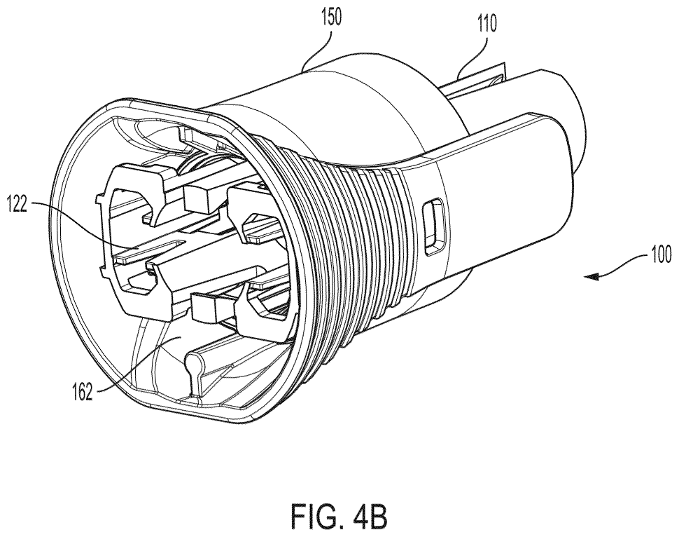

Provided herein is a shield removal assembly for a drug delivery device including a needle. In non-limiting embodiments or aspects, the drug delivery device is an autoinjector or other similar device for automatic or manual delivery of a substance to a user. With reference to FIGS. 1-6, shown is a shield removal assembly (100) including a retainer (110) and a cap remover (150). Retainer (110) is nested within cap remover (150). In non-limiting embodiments or aspects, cap remover (150) is flared at a distal end thereof to form an ergonomic gripping surface to allow a user to firmly grip the cap remover. Retainer (110) and cap remover (150) can be formed of any rigid material, for example, and without limitation, plastics and the like.

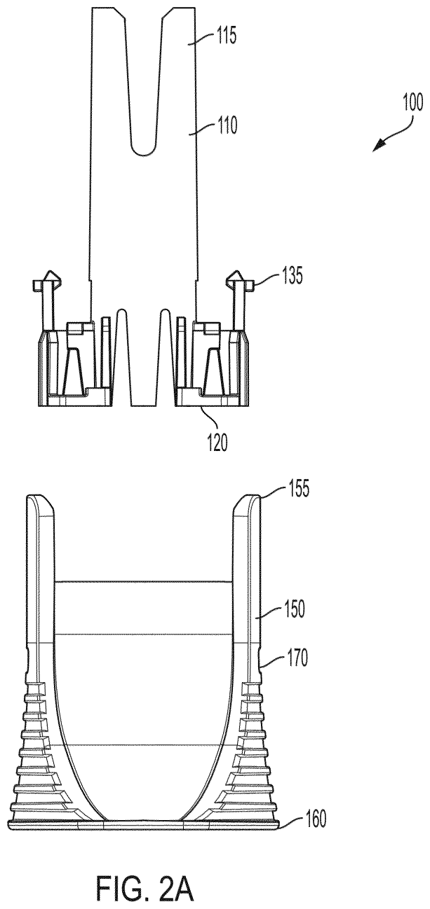

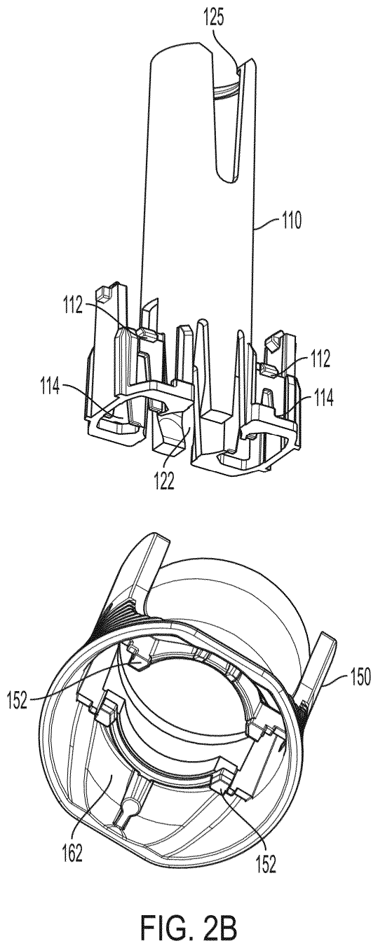

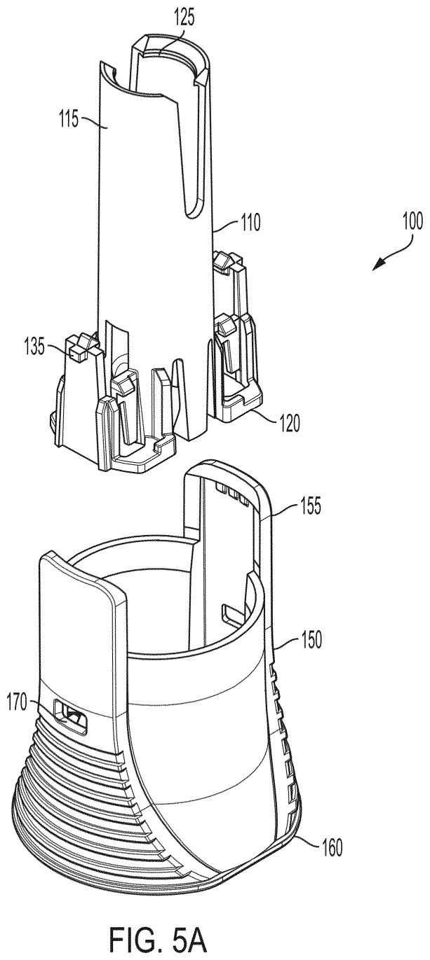

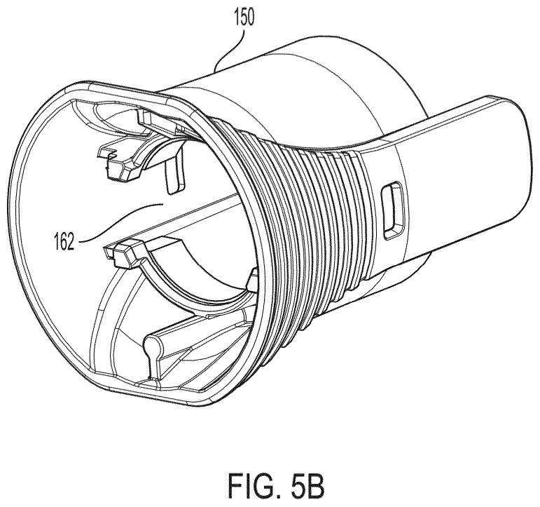

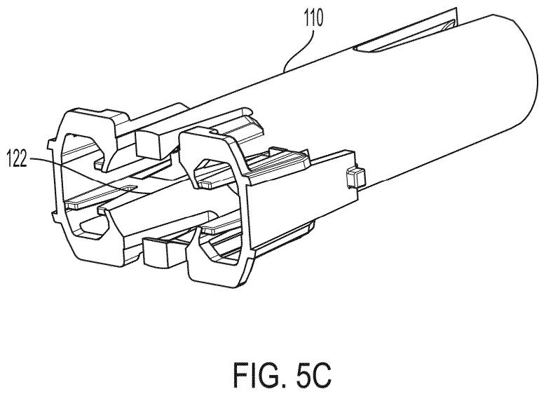

FIGS. 2A, 2B, and 5A-5C show exploded views of a non-limiting embodiment or aspect of the shield removal assembly described herein. Cap remover (150) includes proximal end (155), which is configured to be closest to the housing, and a distal end (160). In non-limiting embodiments or aspects, proximal end (155) of cap remover (150) includes two or more arms that engage with a housing (not shown) of an autoinjector. In such non-limiting embodiments or aspects, the arms of the cap remover (150) include at least one projection or shoulder to allow secure, releasable engagement with the housing of the autoinjector. The distal end (160) of cap remover (150) includes at least one opening (162) therethrough. In non-limiting embodiments or aspects, cap remover (150) includes in a sidewall thereof at least one opening (170). In non-limiting embodiments or aspects, cap remover (150) includes two openings (170). In non-limiting embodiments or aspects, cap remover (150) includes two openings (170) that are located on opposite sides of the cap remover (150).

With continued reference to FIGS. 1, 2A, 2B, 4, 5A, and 5B, retainer (110) includes proximal (115, closest to the housing) and distal (120) ends. In non-limiting embodiments or aspects, proximal end (115) of retainer (110) includes two or more arms that engage with the needle shield (not shown) of an autoinjector. In such non-limiting embodiments or aspects, the arms of the retainer (110) include at least one projection to allow secure engagement with the needle shield. The distal end (120) of retainer (110) includes at least one opening (122) therethrough. In non-limiting embodiments, the distal end (120) of the retainer (110) is configured to abut the distal end (160) of the cap remover (150). In non-limiting embodiments or aspects, the opening (162) in the distal end (160) of the cap remover (150) is of a larger diameter than the opening (122) in the distal end (120) of the retainer (110). In non-limiting embodiments or aspects, this difference in diameter allows the retainer (110) to be introduced into the cap remover (150) through the opening (162) in the distal end (160) of the cap remover (150).

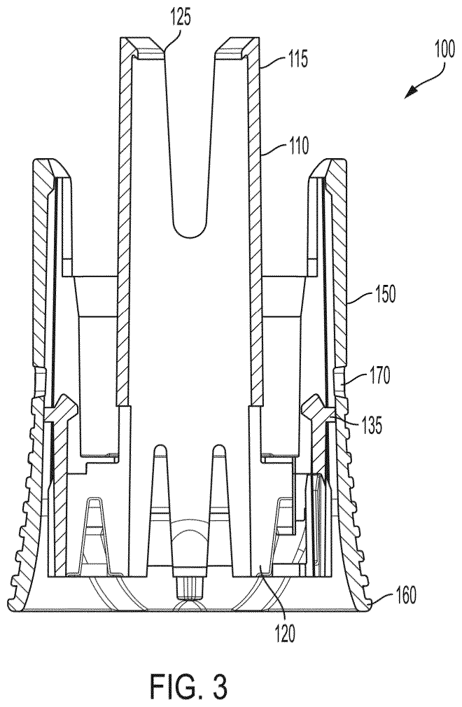

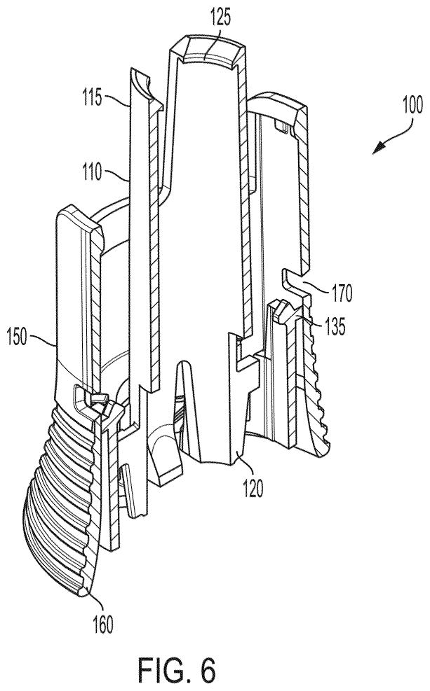

As described previously, proximal end (115) of retainer (110) can include at least one grasping feature, such as a projection, to allow secure engagement with a needle shield. Turning to FIGS. 3 and 6, shown is a shield removal assembly according to a non-limiting embodiment or aspect, where the retainer (110) includes at least one grasping feature (125) at a proximal end (115) thereof. The at least one grasping feature can be at least one protrusion (125), and can be configured such that it is capable of gripping a needle shield by a flange thereof.

In non-limiting embodiments or aspects, the retainer (110) and cap remover (150) of the shield removal assembly (100) are configured or arranged such that the needle shield and/or syringe of an autoinjector (not shown) can be shifted relative to the retainer by introducing a device, such as a tool, through the openings (122, 162) in the distal ends (120, 160) of the retainer (110) and cap remover (150), respectively. Placing a tool through the openings in the cap remover and retainer allows the needle shield and syringe to be displaced proximally, and thus allows for adjustment of the positioning of the projections (125) relative to the needle shield to ensure a secure grip between the retainer (110) and the needle shield.

With continued reference to FIGS. 3 and 6, as described above in non-limiting embodiments or aspects the cap remover (150) includes at least one opening (170) in a sidewall thereof. The at least one opening (170) is configured to allow at least one radially-extending protrusion (135) on retainer (110) to extend therethrough. In the non-limiting embodiment or aspect shown in FIGS. 3 and 6, at least one radially-extending protrusion (135) extends from an arm that extends proximally from the distal end (120) of the retainer (110). In other non-limiting embodiments or aspects (not shown), the at least one radially-extending protrusion (135) is positioned on a main body of the retainer (110).

With reference to FIGS. 2A and 2B, a lag arrangement is provided between the cap remover (150) and the retainer (110) during removal of the shield removal assembly (100). Retainer (110) is received within cap remover (150) such that at least one protrusion (112) is arranged proximally of at least one shoulder (152) of cap remover (150). In non-limiting embodiments or aspects, the at least one protrusion (112) prevents the retainer (110) from being displaced distally out of the cap remover (150), by virtue of engagement with a proximal surface of the at least one shoulder (152). In non-limiting embodiments or aspects, the retainer (110) further includes at least one flange (114), arranged distally of the at least one shoulder (152) of the cap remover (150). In this way, the retainer (110) is prevented from being displaced proximally out of the cap remover (150) by virtue of engagement of a proximal surface of the at least one flange (114) with a distal surface of the at least one shoulder (152). The longitudinal space between the at least one protrusion (112) and the at least one flange (114) on the retainer (110) results in a predetermined amount of “play” between these components. Accordingly, in non-limiting embodiments or aspects, when the cap remover (150) is pulled distally to remove the shield removal assembly (100) from a drug delivery device to which it is attached, cap remover (150) is pulled a certain distance before the at least one shoulder (152) engages the at least one protrusion (112) of the retainer (110). This results in a lag between the initiation of movement of the cap remover (150) and initiation of movement of the retainer (110). When cap remover (150) moves sufficiently such that the at least one shoulder (152) engages with the at least one protrusion (112), the retainer (110) begins to be pulled away from the drug delivery device, and, because of the positioning of the at least one projection (125) relative to the needle shield, the needle shield begins to be pulled away from the syringe and needle.

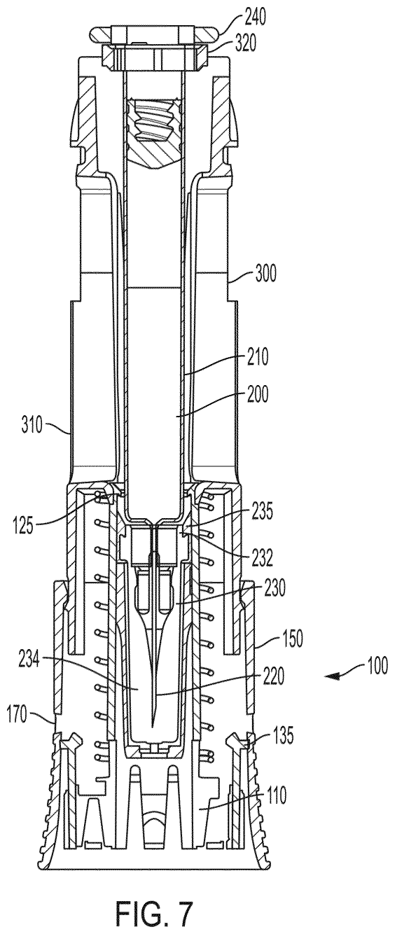

Also provided herein is an autoinjector including a housing, a syringe having a barrel, a needle, and a needle shield, and a shield removal assembly. Turning to FIGS. 7-9, shown is an autoinjector having a housing, a syringe (200) having a barrel (210) and a needle (220) attached thereto, a removable needle shield (230) configured to at least partially surround the needle (220), and a shield removal assembly (100). The autoinjector housing (a lower housing (310) is shown, though it should be understood that autoinjector can include an upper housing, as described below) includes a proximal end and a distal end, where the distal end is open to permit the needle (220) to pass therethrough to allow a substance to be delivered to a user.

Removable needle shield (230) can include proximal (232) and distal (234) ends. In non-limiting embodiments or aspects, removable needle shield (230) is a two-piece needle shield, including an elastomeric inner portion and a rigid outer portion. In non-limiting embodiments or aspects, the rigid outer portion at least partially surrounds the elastomeric inner portion of the needle shield (230). In non-limiting embodiments or aspects, needle shield (230) includes at least one flange or shoulder (235). In non-limiting embodiments or aspects, the at least one flange or shoulder (235) is located on the elastomeric inner portion, for engaging the rigid outer portion. In non-limiting embodiments or aspects, needle shield (230) includes at least one complementary grasping feature (233) on the proximal end (232) thereof. In non-limiting embodiments or aspects, the at least one complementary grasping feature (233) is configured to be complementary to, and engageable with, the at least one grasping feature (125) of the retainer. In non-limiting embodiments or aspects, the at least one complementary grasping feature (233) is the proximal edge of the needle shield (230).

In non-limiting embodiments or aspects, the elastomeric portion of the needle shield (230) is formed of one or more natural or synthetic rubbers and/or of thermoplastic elastomer, or combinations thereof. In non-limiting embodiments or aspects, the rigid outer portion of the needle shield (230) is formed of a rigid plastic. In non-limiting embodiments or aspects, the rigid outer portion is formed of polypropylene.

Shield removal assembly (100) includes, as described previously, cap remover (150) and retainer (110). In non-limiting embodiments or aspects, the cap remover (150) and/or retainer (110) include one or more of the features described above. In non-limiting embodiments or aspects, cap remover (150) at least partially surrounds the open distal end of the housing. In non-limiting embodiments or aspects, cap remover (150) is not rotatable relative to the housing. Autoinjector housing and/or cap remover (150) can include suitable features to prevent rotation of the cap remover (150), and, accordingly, retainer (110) and needle shield (230), relative to the housing and the needle (220), during needle shield (230) removal. In non-limiting embodiments or aspects, suitable anti-rotation features include one or more projections, guide channels, and/or shoulders on cap remover (150) and/or housing.

Also provided herein is a method of assembling an autoinjector including a shield removal assembly, including the steps of providing a lower housing assembly of an autoinjector, inserting a syringe into the lower assembly, and placing an upper assembly on the lower assembly. In a first step, a lower housing assembly (300), including shield removal assembly (100) engaged therewith, is provided. The lower housing assembly (300) includes lower housing (310). In non-limiting embodiments or aspects, lower assembly (300) includes a ring (320) at a proximal end thereof. The lower housing assembly (300) includes a proximal end and a distal end, the distal end being open to allow a needle of a syringe to pass therethrough, and the proximal end of the lower housing (310) being open to allow a syringe to be inserted into the housing. Housing may be formed of any suitable material, including, for example and without limitation, a rigid plastic. Lower assembly (300) also includes a shield removal assembly (100). Shield removal assembly (100) includes, as described previously, cap remover (150) and retainer (110), each having distal ends (160, 120) having an opening (162, 122) therethrough. In non-limiting embodiments or aspects, the cap remover (150) and/or retainer (110) include one or more of the features described above. In an initial configuration (e.g., when shield removal assembly (100) is positioned on lower housing (300)), the distal end (120) of the retainer (110) is spaced from the distal end (160) of the cap remover (150).

With reference to FIG. 7, in non-limiting embodiments or aspects, in a second step of the method, a syringe (200) is inserted into the lower housing (310). The syringe (200) can include a barrel (210) to hold a medicament or other substance therein, a needle (220) arranged at a distal end of the barrel (210), and a removable needle shield (230). Syringe (200), barrel (210), needle (220), and needle shield (230) can include one or more of the features described above. Syringe barrel (210) can be of any useful size, and can be formed of any useful material, such as glass or plastic. In non-limiting embodiments or aspects, syringe (200) is a unitary syringe, and the needle (220) is formed with the syringe barrel (210). In non-limiting embodiments or aspects, syringe barrel (210) is not formed with needle (220) therein and, instead, a needle hub (not shown) holds needle (220). Syringe needle (220) can be formed of any useful material, and can be of any suitable gauge. Needle shield (230) can be a one-piece needle shield formed of an elastomeric material, or a two-piece needle shield having an inner portion formed of an elastomeric material and an outer portion formed of a rigid material. The needle shield (230) at least partially surrounds the needle (220). In non-limiting embodiments or aspects, syringe (200) includes a flange (240) at a proximal end thereof. At the conclusion of the second step, the distal end (120) of the retainer (110) remains longitudinally spaced from the distal end (160) of the cap remover (150).

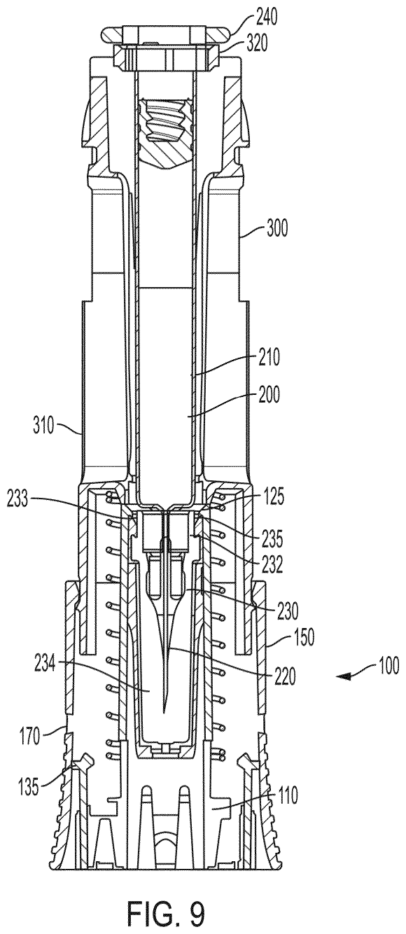

In non-limiting embodiments or aspects, the step of inserting the syringe (200) includes moving the syringe (200) towards the distal end of the housing (310), preferably such that the at least one grasping feature (125) is spaced longitudinally from the proximal end of the needle shield. In other words, the at least one grasping feature (125) is more proximal than in its final position (shown in FIG. 9). In this position, the distal end (234) of the syringe flange abuts the ring (320). In this position, the proximal end (115) of the retainer (110) contacts the syringe barrel (210).

As described above, at the conclusion of this step, the distal end (120) of the retainer (110) is maintained proximally with respect to the distal end (160) of the cap remover (150), such that the distal end (120) of the retainer (110) is longitudinally spaced from the distal end (160) of the cap remover (150). In non-limiting embodiments or aspects, a tool (not shown) is inserted within the opening (162) of the cap remover (150). This tool can be used to push the retainer (110) proximally, such that the distal end (120) of the retainer (110) is longitudinally spaced from the distal end (160) of the cap remover (150).

In non-limiting embodiments or aspects, the proximal end (115) of the retainer (110) includes at least one grasping feature, such as projections (125), and the projections (125) contact the syringe barrel (210) proximally of the distal end of the syringe barrel (210). In this way, the retainer (110) is in an “overstroked” position, where the at least one grasping feature (125) is located too far proximally of the needle shield (230) (FIG. 7). In non-limiting embodiments or aspects, the needle shield (230) includes at least one flange or shoulder (235), and the proximal end (115) of the retainer (110) includes at least one grasping feature (125), and the at least one grasping feature (125) contacts the syringe barrel (210) proximally of the at least one flange or shoulder (235).

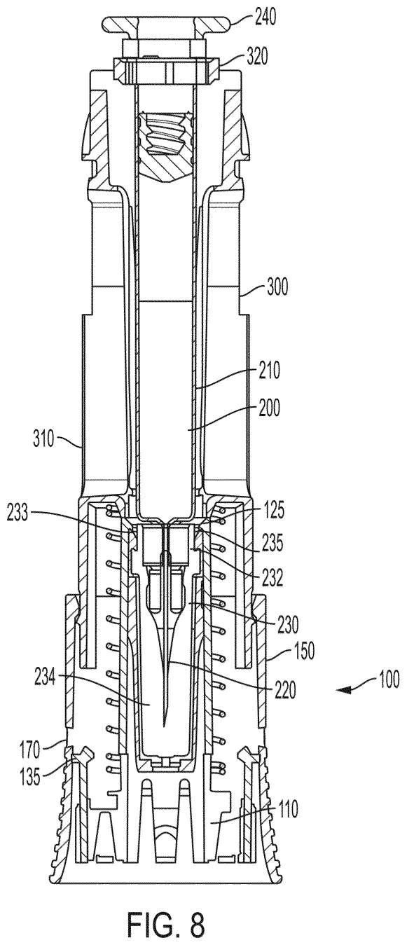

With reference to FIG. 8, in non-limiting embodiments or aspects, a third step of the method of assembling the syringe includes, after inserting the syringe (200) into the lower housing (310), moving the syringe (200) proximally. In non-limiting embodiments or aspects, moving the syringe (200) proximally within the lower housing (310) is achieved by inserting a device through the at least one opening (162, 122) in the distal ends (160, 120) of the cap remover (150) and retainer (110), and applying pressure to urge the syringe (200) proximally. In non-limiting embodiments or aspects, and as shown in FIG. 8, when the syringe (200) is urged proximally, the at least one grasping feature (125) of the retainer (110) engages the needle shield at a proximal-most end thereof. In non-limiting embodiments or aspects, the at least one grasping feature (125) engages at least one corresponding, complementary grasping feature (233) on the needle shield (230). In non-limiting embodiments or aspects, after the syringe (200) is urged proximally, the syringe flange (240) is longitudinally spaced from the ring (320) of the lower assembly (300). At the end of this step the at least one grasping feature (125) engages the at least one complementary grasping feature (233) of the needle shield (230), the distal end (120) of the retainer (110) is longitudinally spaced from the distal end (160) of the cap remover (150), and the syringe flange (240) is longitudinally spaced from the ring (320).

In non-limiting embodiments or aspects, the method includes a fourth step of placing an upper assembly (not shown), including an upper housing, on the lower assembly. As with lower assembly and lower housing, upper assembly and upper housing can be formed of any suitable material, including, for example and without limitation, a rigid plastic. In non-limiting embodiments or aspects, the step of placing the upper assembly on the lower assembly causes the syringe to move distally, such that the syringe flange (240) abuts the ring (320).

With reference to FIG. 9, after assembly, distal end (120) of retainer (110) falls or is otherwise displaced towards the distal end (160) of the cap remover (150), such that the distal end (120) of the retainer (110) abuts the distal end (160) of the cap remover (150).

Once a final user wants to remove the needle shield, he/she pulls the cap remover. As described above, the gap between the at least one protrusion (112) of the retainer (110) and the at least one shoulder (152) of the cap remover (150) causes a lag between the initiation of longitudinal movement of the cap remover (150) away from the lower housing assembly (300) and the initiation of longitudinal movement of the retainer (110) away from the lower housing assembly (300).

Although the invention has been described in detail for the purpose of illustration based on what is currently considered to be the most practical and preferred embodiments, it is to be understood that such detail is solely for that purpose and that the invention is not limited to the disclosed embodiments, but on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A shield removal assembly for an autoinjector comprising a syringe barrel, a needle, and a needle shield at least partially surrounding the needle, the shield removal assembly comprising:
a cap remover configured to be grasped by a user and having a proximal end and a distal end, the distal end of the cap remover comprising at least one opening therethrough; and
a retainer received within the cap remover and having a proximal end and a distal end, the retainer comprising at least one grasping feature configured to engage at least one complementary grasping feature of the needle shield, and the distal end comprising an opening therethrough,
wherein the retainer is axially moveable with respect to the cap remover, and
wherein the retainer comprises at least one radially-extending protrusion, and the cap remover comprises at least one window configured to receive the radially-extending protrusion.

2. The shield removal assembly of claim 1, wherein the at least one grasping feature comprises at least one protrusion, and the at least one complementary grasping feature comprises a proximal edge of the needle shield.

3. The shield removal assembly of claim 1, wherein the shield removal assembly is configured such that the needle shield of the autoinjector can be advanced through the shield removal assembly from a first position in which the at least one grasping feature is spaced longitudinally and located proximally of the at least one complementary grasping feature of the needle shield to a second position in which the at least one grasping feature engages the at least one complementary grasping feature of the needle shield.

4. The shield removal assembly of claim 1, wherein the at least one grasping feature is located at the proximal end of the retainer, the shield removal assembly configured such that the needle shield of the autoinjector can be advanced through the shield removal assembly from a first position in which the proximal end of the needle shield is longitudinally spaced from the at least one grasping feature of the retainer to a second position in which the proximal end of the needle shield abuts the at least one grasping feature of the retainer.

5. The shield removal assembly of claim 1, wherein:
the retainer comprises at least one protrusion; and
the cap remover comprises at least one shoulder longitudinally spaced from the at least one protrusion of the retainer,
such that the cap remover is moved longitudinally away from the syringe a set distance before the at least one shoulder engages the at least one protrusion.

6. The shield removal assembly of claim 1, wherein the opening in the distal end of the cap remover has a greater diameter than the opening in the distal end of the retainer.

7. An autoinjector comprising:
a housing having a proximal end, an open distal end, and defining a longitudinal axis;
a syringe held within the housing and having a barrel;
a needle attached to a distal end of the syringe barrel;
a removable needle shield having a proximal end and a distal end, the needle shield configured to at least partially surround the needle; and
a shield removal assembly according to claim 1.

8. The autoinjector of claim 7, wherein the cap remover is not rotatable relative to the housing.

9. A method of assembling an autoinjector comprising:
providing a lower assembly, the lower assembly comprising:
a lower housing having a proximal end and a distal end, and defining a longitudinal axis; and
a shield removal assembly arranged at the distal end of the lower housing, comprising:
a cap remover configured to be grasped by a user and having a proximal end, a distal end, the distal end of the cap remover comprising at least one opening therethrough; and
a retainer received within the cap remover and having a proximal end comprising at least one grasping feature and a distal end, the distal end of the retainer comprising at least one opening therethrough and spaced from the distal end of the cap remover in an initial configuration;
inserting a syringe into the lower assembly, the syringe comprising:
a barrel having a proximal end and a distal end;
a needle arranged at the distal end of the barrel; and
a removable needle shield having a proximal end comprising at least one complimentary grasping feature and a distal end, the needle shield configured to at least partially surround the needle,
wherein the step of inserting comprises moving the syringe towards the distal end of the lower housing along the longitudinal axis such that the at least one grasping feature of the retainer is spaced longitudinally and located proximally of the at least one complementary grasping feature of the needle shield; and
placing an upper assembly comprising an upper housing on the lower assembly.

10. The method of claim 9, wherein the at least one grasping feature of the retainer is a projection configured to engage a proximal edge of the needle shield, and wherein the step of inserting the syringe into the lower assembly comprises urging the syringe distally such that the at least one projection is positioned proximally and is longitudinally spaced from the proximal edge of the needle shield.

11. The method of claim 10, further comprising a step of,
after inserting the syringe into the lower assembly, urging the syringe proximally such that the at least one grasping feature engages the proximal edge of the needle shield.

12. The method of claim 11, wherein the step of urging the syringe proximally comprises inserting a device through the at least one opening in the distal end of the cap remover and the opening in the distal end of the retainer to urge the syringe proximally.

13. The method of claim 10, wherein the proximal end of the syringe barrel comprises a flange and the proximal end of the lower housing comprises a ring.

14. The method of claim 13, wherein, after insertion of the syringe, the syringe flange abuts the ring.

15. The method of claim 14, further comprising, after inserting the syringe into the lower assembly, a step of urging the syringe proximally, and wherein after the step of urging the syringe proximally, the syringe flange is longitudinally spaced from the ring.
